(19) United States
(12) Patent Application Publication
(10) Pub. No.: US 2023/0088645 A1
(43) Pub. Date: Mar. 23, 2023

(54) Intelligent Preemptive Medical Screening

(71) Applicant: Dov Biran, Scarsdale, NY (US)

(72) Inventor: Dov Biran, Scarsdale, NY (US)

(21) Appl. No.: 17/478,908

(22) Filed: Sep. 18, 2021

(51) Int. Cl. G16H 50/30; G16H 10/60; G16H 10/20; G16H 50/70

(52) U.S. Cl. CPC G16H 50/30; G16H 10/60; G16H 10/20; G16H 50/70

(57) ABSTRACT

This document describes a system that combines medical screenings capabilities for multiple conditions and disease with dynamic alerting and learning mechanisms. Via digital questionnaires and surveys, clinical patients utilize the system as a risk-assessment tool which classifies individuals' likelihood to have or develop a certain condition. Real-time dynamic alerting reacts to patients' answers with actionable information when high-risk classification is observed from a patient's responses. Historical medical records are fed into a feedback loop which improves the system's risk assessment by learning previous survey answers that correlate to a patient with specific positive diagnosis of the screened condition. These intelligent screening procedures define the main purpose of the invention, and integrate with other healthcare resources such as doctor consultations and appointment scheduling to give medical providers as well as patients a comprehensive screening functionality that spans several levels in the medical digital technology vertical.

### TECHNICAL FIELD

The invention relates to the field of healthcare, telemedicine, and information systems. Specifically, it relates to digital screening methodologies in medical settings with an application of statistical learning and artificial intelligence.

### BACKGROUND ART

The medical screening process requires a patient to complete a set of questions, the answers to which are used as an indication of a specific medical condition. The finding, or lack thereof, of such conditions are used as guidelines for further action by the patient. These guidelines can range from the recommendation of further screening to an immediate call for drastic medical treatment.

Digital advances have improved upon screening procedures by allowing computers to adaptively modify the questions a patient has to complete, depending on previous answers they submitted. In this way, the surveys more efficiently assess the risk a patient may be in. However, there are still several areas in which screenings can be improved.

The medical field is much more complex than the simple task of assessing patients for the risk of a condition, as other objectives still need to be met. These non-arbitrary tasks such as sending patients preventive information and scheduling medical visits when they are required are as crucial to the patient's well-being as the screening procedure is. Most importantly, immediately alerting patients of high risk for a medical condition is essential for optimal medical care, and is not possible using modern screening tools. If a patient's answers indicate a serious and even life-threatening condition, an alert to seek immediate care is imperative.

The system described in this document is built to fulfill all these requirements and does so in an innovative manner. Our invention ensures the best possible outcomes for patients while also taking into account the remaining players in the field who exist in the medical care process such as payers and health providers.

### SUMMARY

The preventive aspect of modern medicine is prevalent among a multitude of practices and has been applied to a range of conditions and illnesses, especially with the help of digital technology. Many inventions and innovations in medicine have been focused on identifying at-risk patients as early as possible, using dynamic questionnaires and entrenching fast-acting computer-implemented processes into the medical care of individuals. Patients' unique responses to digital surveys and questionnaires give medical professionals an easy form of early diagnosis, and provide the patients with preliminary and early intervention and care.

However, these advancements lack in many areas and aspects. First, they are not robust enough to support a diverse and wide-spread vertical network of payers, consumers and caretakers in the medical field. The utilization of digital technology in these advancements is also lackluster in automating many of the necessary medical processes and alerting patients based on their information and background. Modern tools in data analysis and machine learning can also provide further improvements onto these digital innovations, and intelligently identify at-risk cohorts using validated past assessments and new patient data. New innovation in the space can significantly bolster the efficacy and performance of screening methodologies and patient care tools.

The innovation described in this document comprises an overarching tool made of these precise methods, and entails an invention meant to improve the outcomes for both patients, clinicians and insurers in the medical field. When applied onto a large population of patients, the system would provide its users with a variety of screening options, each specific to a condition or disease that may be prevalent among a specific sub-population. Members of this group would be prompted with a dynamic screening questionnaire which, based on patient answers, can react in real time with alerts and automatic enrollment in Action Plans (BRAHMS). Additionally, past survey entries and diagnoses are used by the system to improve these functionalities, and patient data that matches positive screening for a condition can be used to better identify which current individuals are at high risk. These intelligent methodologies, in combination with the questionnaire answers and the screened patient's personal data, are used to assign each patient a risk category and appropriate care professionals. A feedback loop which takes clinician diagnosis to validate the risk assessment is used to improve future classifications by the system, and feeds a learning algorithm which increases the invention's efficacy of finding high risk individuals. The system also classifies cohorts into moderate and low risk groups and automatically presents such individuals with educational material, and information about the condition they may develop high risk for in the future. The invention would provide patients with the opportunity to schedule an appointment with professionals directly after being screened. A reporting functionality that gathers all of the information produced and processed by the invention, such as patient classification and risk assessment results, would be available for the users to review the systems functionality and performance. The overarching goal of the system, screening a population of patients for a potential condition or disease, is made seamless by digital automation yet still tailored to the patient by using personal data to arrive at accurate and actionable assessments.

The screening campaigns made possible by the invention can be managed by the system, executed instantaneously and reviewed after-the-fact. Future campaigns can be referenced and guided by the results of past campaigns, which are stored and reported-on by the invention's many capabilities.

### DESCRIPTION OF DRAWINGS

FIG. 1: A flow chart illustrating the screening process in its entirety.

FIG. 2: An illustration of the connection between the invention and its users, as a deployment over a network of computers and servers.

FIG. 3: A diagram showing the different modules and repositories that comprise the system.

FIG. 4: A flow diagram representing the dynamic nature of screening questionnaire creation by the system.

FIG. 5: A flow diagram showing the analytical and learning processes that follow the screening process when assessment validation information is available.

### DETAILED DESCRIPTION

### System Overview

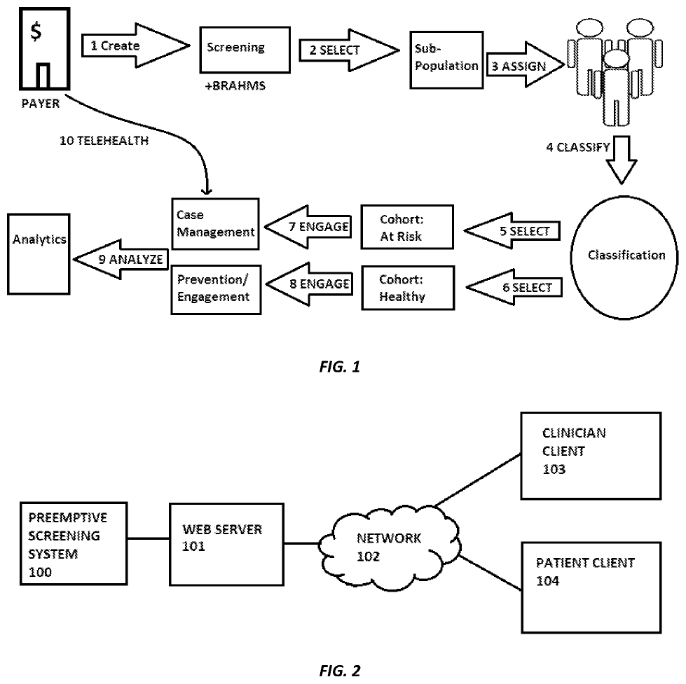

In FIG. 1, the main objective and processes of the system are shown in a sequential flow chart describing the distinct parts comprising the invention. At the top left, payers such as health insurance companies create a screening procedure for individuals. This selection is illustrated by procedure 1 in the image. Using the system, the payers would have a large selection of screening choices, varying from mental conditions such as depression to more severe chronic diseases like cancer. These distinct screenings would consist of questions that are unique to the condition. Each survey would also be accompanied by BRAHMS capabilities, which could trigger alerts and automatic actions depending on the patient's answers. For example, if a patient answeres positively for a question that indiciates an immidiate risk of suicide or other grave medical dangers, the BRAHMS system can alert the patient as well as a medical professional on the risk for the individual, and immidiately act upon the risk. The entire screening survey would be applied to the selected sub-group of the population, which is illustrated by procedure 2 in the image. The questionnaire would be distributed by the system to the entire subpopulation, which can be selected under a variety of scenarios, such as employees of a certain company, or patients in a specific hospital or clinic. As they complete the survey, the system would track the probability of each individual to be at risk for the screened condition. At the point where this risk has been identified or disproven, the system classifies the individual accordingly. This classification may occur at the completion of the questionnaire, or earlier by the BRAHMS system. For example, if a patient has only completed half of the survey, but their answers have so far given a significant indication of risk, the remainder of the questionnaire may not need to be completed, and classification can be made early, terminating the screening procedure prior to receiving all the answers. Alternatively, an individual may answer the complete questionnaire and be found to have no risk for the screened condition, in which case the classification of low risk would be done at the end of the screening process. In both classification timings, the classification is considered to be a singular piece of the invention, and is illustrated by procedure 4 in the image. Procedure 5 represents the classification result in the case where the patient has shown enough risk for a positive diagnosis of the screened disease, while procedure 6 is the alternative procedure which gets executed when the patient does not exhibit enough risk to carry the disease. In the former case, the patient would be engaged as if a positive diagnosis was made, and immediate management of the condition would begin. This could be embodied by an alert to a relevant physician or a request for the patient to follow up the screening process with further testing, illustrated by procedure 7 in the image. Following the latter case, a patient with low risk of having the screened condition would be engaged with preventive information, and guidance on how to maintain a low risk for the screened condition via resources such as information packets and relevant publications. This engagement is illustrated by procedure 8 in the image. Following these procedures, patient engagement and other metrics can be analyzed for improvement. This analysis may be used to better understand how patients prefer to be engaged, which information is most relevant and best improves patients' quality of life, or any other form of analysis. Illustrated by procedure 9 in the image, analysis may also consist of validation if patients' actual diagnosis confirms or contradicts the system's risk assessment. In the case that a physical visit with a physician or lab test confirms the system's positive risk assessment, the patient's data and screening results may be analyzed and used to reinforce the classification process. If the physical results contradict the system's classification, revealing false positive assessments, the system may analyze the patient's data to learn how to better classify similar patients in the future, Similarly, the analysis may be done in the case that the system falsely classified a patient to be at low risk for the screened condition. Procedure 10 provides the payers, via data management and visualization processes, a wide view of the medical screening's results. Payers will be able to analyze individual as well as collective screening diagnoses and make decisions regarding future care for their insured. Additionally, payers could control the classification configurations and any other aspect of the screening procedures. Using classical telehealth processes, the payers would be able to complete all of these tasks without requiring the physical presence of any of their insured patients.

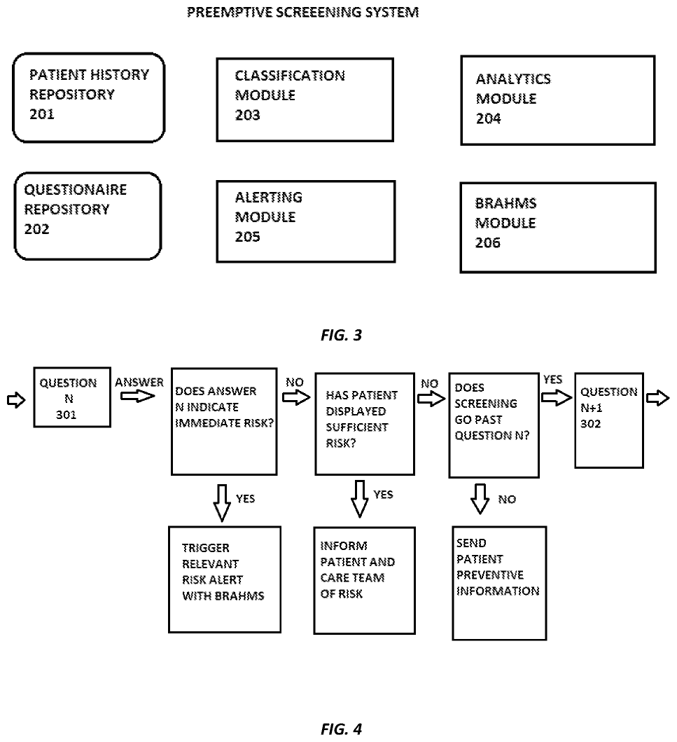

FIG. 2 presents the network 102 which represents the communication pathway between a user and the system 100. A user in this scenario may be a patient 103 accessing the screening procedures, or a medical professional 104 which controls and observes the screening processes. The network can be the internet or other communication lines that are not part of the internet, either one using standard communication protocols.

The web server 101 presents the system 100 to the users using an interface which could be in the form of web pages. The users utilize this interface to provide information to as well as receive information from the system 100. This information could regard any aspect of the system, whether it is patient data, screening surveys, medical information or information about the users. For example, the information could pertain to a patient 104 accessing a screening survey and responding to questions accordingly, relaying information back to the system 100.

The patient's client 104 is used to transmit this information. It can be embodied by any device that can connect to the network 102. This could be a smartphone, PC, laptop or any special purpose processor that can communicate with the web server 101. The interface through which the system 100 is presented can be a web browser such as Google Chrome or Internet Explorer, as well as other special purpose interfaces built for the system 100. More components and modules of the system 100 are described below.

FIG. 3 displays the technical layout of the components comprising the intelligent preemptive medical screening system. Two data stores, one containing patients and their medical histories 201 and another containing the questionnaires and screening surveys 202, make up the backend of the system. These may be deployed on computer servers or any other data storage and management tools.

The BRAHMS module 206 contains the procedures and processes pertaining to the alerting and real-time reactions to patients' screening answers. When using the system, care managers may program and create alerts and reactions within the module as a response to specific answers by patients. For example, if a caregiver or payer is creating a screening survey for depression or other mental health conditions, a BRAHMS alert may be programmed into the survey to alert a professional when a patient answers positively to a question regarding suicide risk. As a patient completes a screening questionnaire, their responses are monitored by the BRAHMS module, which reacts accordingly if specific alerting conditions such as those explained above are met. The module may be deployed on a single or set of digital processors, and may even include within it the code and software which executes the screening processes when accessed by the patients.

The alerting module 205 includes the processes which extend past the BRAHMS and screening system, such as integration with outside players like doctors or other relevant professionals. These relevant players are alerted via the alerting module, which assures that an at-risk patient receives the necessary treatment when triggering a BRAHMS alert. Such triggers may be calling public health responders like 911 or contacting any other immediately available help. Alerts can also respond to patients with instructions, or forward the relevant patient's information to a physician or professional who may be of help.

The classification module 203 executes the processes necessary when a patient completes a screening procedure. Given the responses to the survey questions, a patient's risk of having the screened condition is determined by the procedures programmed into the classification module. Any one of many decision making programs may be used to fulfill the classification module's function, and different programs may be used for different screening procedures. For example, a patient who answers positively to being over 65 years of age may be classified as being at risk for alzheimers after completing a screening for the condition. In contrast, a combination of weighed covariates including age, gender and other medical conditions may be used in a nonlinear binary classifier which determines the risk of a patient to have lung cancer. In either scenario, or any other, the classification module would follow its quantitative computation with a classification of either high or low risk for the screened condition. The result may be forwarded to the patient or only to a caregiver or relevant professional. Alternatively, the classification may be relayed in the form of supplemental material. For example, a high risk classification may be followed by the requirement for the patient to schedule an appointment with a caregiver for further steps. A BRAHMS alert may also be triggered by a positive high risk classification. On the other hand, a low risk classification could be followed by forwarding the screened patient relevant preventive information, to assure that they will not develop the screened condition in the future. Any result may or may not be shared with a relevant caregiver by the classification module. These sub-procedures may be contained within the same program and executed by the same processors, or alternatively be distributed over a network or collection of computers or processors.

The analytical module 204 hosts the processes and procedures which oversee and improve the accuracy and performance of the classification module. Patients may provide feedback in the form of validation or contradictions to the screening procedure's final classification. For example, a patient who was classified to be at high risk of a screened condition may be required to receive further tests and treatments. These tests may result in a contradiction to the system's classification, and show that the patient does not have the condition in reality. Alternatively, the tests may confirm the system's classification. Vice versa, a classification of low risk for a patient may also be confirmed or contradicted after the screening process is complete. The processes hosted by the analytical module take as input these contradictions or validations, and learn how to better classify future patients into risk categories. One possible implementation of the analytical module is to directly connect it to the parameters underlying the classification module. As patient data is aggregated to the patient history repository 201, the analytical module may automatically react by modifying the classification weights and parameters if the newly added information indicates a previous mistake. For example, if a significant number of patients under 65 years of age continue to be incorrectly classified as' being at high risk for alzheimers, the analytical module could reduce the weight or risk-level for this age group when making classification decisions in the future. The analytical module would also be able to produce comprehensive and extensive reports on past screening results. Caregivers and other professionals may use the analytical module and override its learning procedures to make direct changes to the classification processes. This gives users fine control over the false positives and false negatives that the classification module results in.

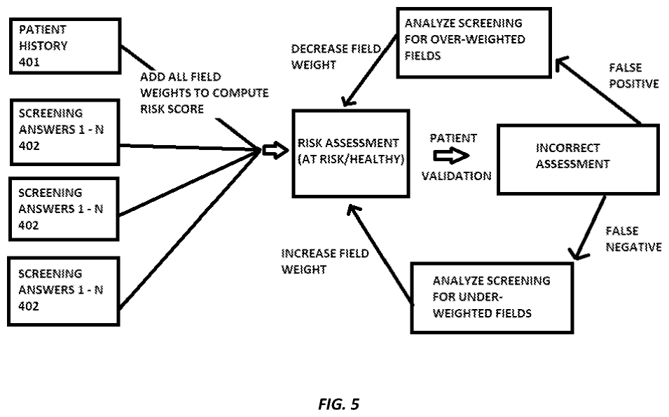

FIG. 4 displays a generic screening process executed by the BRAHMS and alerting modules. Once a screening survey has been opened by a patient using the system, questions from the questionnaire database 202 are added to the survey as displayed by the flow diagram. The patient is prompted with a question 301 and their response is checked by the BRAHMS module for potential alert or immediate action. If an alert or action rule is triggered by the patient's answer, the screening procedure may be stopped, and the patient may be asked or required to act immediately. Alternatively, the alert may be triggered without notifying the patient, and the patient may continue on with the survey. In this case, or if no alert was triggered, the risk score for the patient as computed by the classification module may be checked even if the survey has not yet been completed. It may be the case that the patient's answers so far have indicated enough of a risk to classify a final assessment. If that is the case, the patient may be notified with the risk classification, and prompted with further action steps and instructions. If a high risk classification cannot yet be determined, the next question 302 may be presented to the patient. Otherwise, if the patient had answered the final question in the survey, high risk for the screened condition may not be present and a low risk classification can be made. In this case, relevant preventive information may be presented to the patient, or any other form of feedback that fits the low-risk classification.

FIG. 5 represents the processes executed by the analytical module once a patient has completed a screening survey. First, the patient's history and medical data 401 is used in combination with the patient's questionnaire answers by the classification module to make a determination of high or low risk for the patient. As mentioned before, this determination may be made prior to the patient completing all the answers in the survey. Different survey answers, similar to different patient historical characteristics, may have different weights when computing the singular classification of risk. Once the classification is made and after the patient and relevant professionals are informed, a contradiction or validation to the classification may be available. If the risk assessment was incorrect, one of two scenarios may be at hand. If a positive classification was made and the patient was determined to be at high risk for the screened condition, the system may take the false positive contradiction and analyze its decision in the search for characteristics in the patient's history and survey answers that were weighed too greatly. This weight may be reduced for future classifications, with the goal of reducing false positives. The other scenario of a false negative comes when a patient was determined to be at low risk for the screened condition. After potential follow-up tests, a contradiction to this classification may require the analytical module to look for the under-weighted characteristics. Finding either the over or under weighed characteristics in these scenarios may be done by one of many statistical learning procedures. For example, the analytical module may batch false positive or false negative results and look for common characteristics among these groups. This way, the over or under weighed characteristics may be more easily found. As an example, if a group of patients under the age of 65 were given a false positive classification for being at risk of alzheimers, the analytical module may view the age characteristic as the clear over-weighed characteristic for the screening classification. As a result, the weights would be accordingly adjusted so that the classification module would avoid such mistakes in the future.

1. A computer implemented system which:
a. Stores medical screening questionnaires for multiple conditions as well as medical history data of patients.
b. Allows users to prompt a group of patients with medical screening surveys.
c. Alerts patients and healthcare professionals when a screened patient presents immediate medical risk.
d. Classifies patients into high and low risk categories for the screened condition.
e. Learns from validations and contradictions to its classifications how to better determine patients' risk for a certain condition.
f. Allows users to adjust its classification parameters in order to adjust the total high and low risk classifications made.

2. The method in which claim 1 is implemented, where users may choose to present a single or multiple screening surveys to any group of patients whose information is stored in the system.

3. The method in which claim 1 is implemented, where screening surveys are dynamically built as patients respond to each question individually.

4. The method in which claim 1 is implemented, where a statistical model is used to turn patient data and survey answers into risk classifications.

5. The method in which claim 1 is implemented, where a statistical model is used to improve upon past classifications by learning from validations and contradictions to the system's classification results.

6. The method in which claim 1 is implemented, where screening storage includes relevant preventive information for individuals who may have low risk for the screened condition, as well as relevant referrals to medical tests and instructions for those who may be at high risk for the condition.

7. The method in which claim 1 is implemented, where direct communication lines are implemented from the system to professionals who can immediately react to patients who are at imminent medical danger.

8. A distributable system consisting of computer code and instructions on several processors programmed to:
a. Execute dynamic survey code which adjusts to the questions given by patients.
b. Analyze patient survey answers and history to classify them into high and low risk categories.
c. Adjust its classification mechanism when incorrect determinations are made.
d. Present medical information to patients and professionals based on patients' answers to screening questions.
e. Execute alerts and direct communication protocols to systems outside of the invention when necessary.

9. The method in which claim 7 is implemented, where a new screening process is added to the system.

10. The method in which claim 7 is implemented, where a new group of patients is added to the system.

11. A computer program product running code, the code executed by one of the processors and the operations performed by the code, all consisting of:
a. A database storing medical history data for each patient in the system
b. A database storing screening questionnaires and relevant alerts embedded into the surveys.
c. A module that contains the code and programs executing the classification of patients into high and low risk categories.
d. A module that contains the code and programs executing the analytical computations done to improve the classification based on validation and contradictions of past patient classifications.
e. A module that contains the code and programs observing and reacting to patient survey answers with relevant information and feedback.
f. A module that contains the code and programs executing the alerts and automated responses to patient answers which indicate serious probability for imminent health and medical risk.
